(19) United States
(12) Patent Application Publication
(10) Pub. No.: US 2026/0360496 A1
(43) Pub. Date: Nov. 12, 2026

(54) METHODS AND RELATED ASPECTS FOR MAPPING AND TREATING TUMORS USING ITERATIVE SEED OPTIMIZATION

(71) Applicant: UNM RAINFOREST INNOVATIONS, Albuquerque, NM (US)

(72) Inventors: Stefan POSSE, Albuquerque, NM (US); Kevin M. ROSENBERG, Albuquerque, NM (US)

(21) Appl. No.: 19/487,596

(22) PCT Filed: May 31, 2024
(86) PCT No.: PCT/US2024/031948

§ 371 Date: Nov. 25, 2025

Related U.S. Application Data
(60) Provisional application No. 63/505,462, filed on Jun. 1, 2023.

(51) Int. Cl. G16H 20/10; G06N 20/00; G16H 30/40; G16H 50/70

(52) U.S. Cl. CPC G16H 20/10; G06N 20/00; G16H 30/40; G16H 50/70

(57) ABSTRACT

Provided herein are methods of treating tumors in test subjects. In some embodiments, the methods include predicting a peri-tumoral seed placement proximal to the tumor in a test subject with peri-tumoral connectivity based upon a test subject image data set obtained from the test subject to produce a predicted seed. In some embodiments, the methods also include conducting at least one iteration of a seed optimization protocol that reduces a spatial extent of the predicted seed and that increases a spatial specificity of the peri-tumoral connectivity to produce a segmented test subject image data set. In some embodiments, the methods additionally include administering and/or discontinuing administering one or more tumor therapies to the test subject based at least in part on the segmented test subject image data set. Related systems and computer readable media are also provided.

### CROSS-REFERENCE TO RELATED APPLICATIONS

This application is a U.S. National Phase application of PCT/US2024/031948, filed May 31, 2024, which claims priority to U.S. Provisional Patent Application Ser. No. 63/505,462, filed Jun. 1, 2023, the disclosures of which are hereby incorporated by reference in their entireties.

### STATEMENT OF GOVERNMENT SUPPORT

This invention was made with government support under 1R41NS090691-01A1 and 1R21EB022803-01 awarded by the National Institutes of Health. The government has certain rights in the invention.

### BACKGROUND

Presurgical resting state functional magnetic resonance imaging (rsfMRI) based mapping is an emerging adjunct to task-based functional magnetic resonance imaging (fMRI) for mapping functional connectivity. Seed-based connectivity (SBC) is the most widely used approach for clinical studies since it provides high sensitivity and straightforward interpretation of connectivity in single patients. However, functional neuroanatomy changes in the vicinity of tumors can make seed selection in the vicinity of the tumor unreliable, despite considerable progress with classifier-based brain parcellation and neural network-based training.

Accordingly, there is a need for additional seed selection approaches related to tumor tissue segmentation.

### SUMMARY

This disclosure describes systems, computer readable media, and related methods for treating or guiding treatment of tumors that utilize a model-free seed selection approach using deep learning-based tumor tissue segmentation in combination with iterative subject-specific seed-optimization which improves the specificity of peri-tumoral seed selection. In some embodiments, the methodology automates seed placement in the vicinity of the tumor in the zone that is at risk during surgical resection without relying on neurofunctional brain atlases. As described herein, for example, the evaluation of cortical eloquence in different tumor subregions, such as edematous and infiltrative regions is feasible using a single MRI contrast. In some embodiments, this computationally efficient approach is integrated into a real-time resting-state fMRI analysis pipeline to characterize peri-tumoral connectivity in patients with glioblastomas. In some embodiments, the methods and other aspects of the present disclosure are directed at mapping eloquent cortex using resting-state functional MRI (rsfMRI). In some embodiments, treatment of tumors includes surgical resection and radiation treatment that avoids eloquent cortex, guided by rsfMRI. These and other aspects will be apparent upon a complete review of the present disclosure, including the accompanying figures.

In one aspect, the present disclosure provides a method of treating or guiding treatment of a tumor in a test subject. The method includes predicting, by a computer, a peri-tumoral seed placement proximal to the tumor in the test subject with peri-tumoral connectivity based upon a test subject image data set obtained from the test subject to produce a predicted seed. The method also includes conducting, by the computer, at least one iteration of a seed optimization protocol that reduces a spatial extent of the predicted seed and that increases a spatial specificity of the peri-tumoral connectivity to produce a segmented test subject image data set. In addition, the method also includes administering and/or discontinuing administering one or more tumor therapies to the test subject based at least in part on the segmented test subject image data set, thereby treating the tumor in the subject.

In some embodiments, the test subject image data set comprises a magnetic resonance imaging (MRI) data set. In some embodiments, the seed optimization protocol comprises a model-free seed selection process. In some embodiments, the seed optimization protocol comprises a machine learning technique. In some embodiments, a cranium of the test subject comprises the tumor. In some embodiments, the tumor comprises a glioblastoma. In some embodiments, the method does not utilize a neurofunctional brain atlas.

In another aspect, the present disclosure provides a system for producing a tumor therapy recommendation for a test subject having a tumor using a test subject image data set obtained from the test subject. The system includes a processor; and a memory communicatively coupled to the processor, the memory storing instructions which, when executed on the processor, perform operations comprising: predicting a peri-tumoral seed placement proximal to the tumor in the test subject with peri-tumoral connectivity based upon the test subject image data set obtained from the test subject to produce a predicted seed; conducting at least one iteration of a seed optimization protocol that reduces a spatial extent of the predicted seed and that increases a spatial specificity of the peri-tumoral connectivity to produce a segmented test subject image data set; and, generating a tumor therapy recommendation report that comprises one or more recommendations to administer and/or discontinue administering one or more tumor therapies to the test subject based at least in part on the segmented test subject image data set.

In some embodiments, the test subject image data set comprises a magnetic resonance imaging (MRI) data set. In some embodiments, the seed optimization protocol comprises a model-free seed selection process. In some embodiments, the seed optimization protocol comprises a machine learning technique. In some embodiments, the seed optimization protocol utilizes an electronic neural network. In some embodiments, a cranium of the test subject comprises the tumor. In some embodiments, the tumor comprises a glioblastoma. In some embodiments, the system does not utilize a neurofunctional brain atlas.

In another aspect, the present disclosure provides a computer readable media comprising non-transitory computer executable instructions which, when executed by at least one electronic processor, perform at least: predicting a peri-tumoral seed placement proximal to tumor in a test subject with peri-tumoral connectivity based upon a test subject image data set obtained from the test subject to produce a predicted seed; conducting at least one iteration of a seed optimization protocol that reduces a spatial extent of the predicted seed and that increases a spatial specificity of the peri-tumoral connectivity to produce a segmented test subject image data set; and, generating a tumor therapy recommendation report that comprises one or more recommendations to administer and/or discontinue administering one or more tumor therapies to the test subject based at least in part on the segmented test subject image data set.

In some embodiments, the test subject image data set comprises a magnetic resonance imaging (MRI) data set. In some embodiments, the seed optimization protocol comprises a model-free seed selection process. In some embodiments, the seed optimization protocol comprises a machine learning technique. In some embodiments, a cranium of the test subject comprises the tumor. In some embodiments, the tumor comprises a glioblastoma. In some embodiments, the computer readable media does not utilize a neurofunctional brain atlas.

### BRIEF DESCRIPTION OF DRAWINGS

The above and/or other aspects and advantages will become more apparent and more readily appreciated from the following detailed description of examples, taken in conjunction with the accompanying drawings, in which:

FIG. 1 is a flow chart that schematically shows exemplary method steps of treating a tumor in a test subject according to some aspects disclosed herein.

FIG. 2 is a schematic diagram of an exemplary system suitable for use with certain aspects disclosed herein.

FIGS. 3A-3C show tumor segmentation in a patient with a glioblastoma from the BraTS'21 database. (a) Ground truth. (b) Prediction based on four MRI contrasts available in BraTS'21: encircled area with solid parallel lines slanted downward to the right=edema, encircled area with dashed parallel lines slanted downward to the left=enhancing tumor, encircled area with solid parallel lines slanted downward to the left=non-enhancing/necrotic tumor. (c) Prediction based on T₂ only.

FIGS. 4A-4E show peri-tumoral seed placement in patient with glioblastoma using deep learning-based tissue prediction. (a) Presurgical T2 MRI (b) Prediction of peri-tumor edema and dilation region (encircled area with alternating solid and dashed parallel lines slanted downward to the right) based on presurgical T2 MRI with presurgical peri-tumoral connectivity (encircled area with alternating thicker and thinner solid parallel lines slanted downward to the right). (c) Single-iteration seed optimization strongly reduces spatial extent of seed (encircled area with alternating solid and dashed parallel lines slanted downward to the right) with respect to (b) and increases spatial specificity of peri-tumoral connectivity (encircled area with alternating thicker and thinner solid parallel lines slanted downward to the right). (d) Resting-state localization of sensorimotor network. (e) task-activation in motor area.

FIGS. 5A-5F show peri-tumoral seed placement in 2nd patient with glioblastoma using deep learning-based tissue prediction. (a) Intraoperative view of tumor (b) ECS localization of language stimulation (crosshair) overlaid on T2 MRI (c) Prediction of peri-tumor edema and dilation region (encircled area with alternating solid and dashed parallel lines slanted downward to the right) based on T2 MRI. (d) Single-iteration seed optimization strongly reduces spatial extent of seed (encircled area with alternating solid and dashed parallel lines slanted downward to the right) with respect to (c) and increases spatial specificity of peri-tumoral connectivity (encircled area with solid parallel lines slanted downward to the left). Localization of Exner's area in (e) resting-state fMRI and (f) during verb generation.

### DEFINITIONS

In order for the present disclosure to be more readily understood, certain terms are first defined below. Additional definitions for the following terms and other terms may be set forth throughout the specification. If a definition of a term set forth below is inconsistent with a definition in an application or patent that is incorporated by reference, the definition set forth in this application should be used to understand the meaning of the term.

As used in this specification and the appended claims, the singular forms “a,” “an,” and “the” include plural references unless the context clearly dictates otherwise. Thus, for example, a reference to “a method” includes one or more methods, and/or steps of the type described herein and/or which will become apparent to those persons skilled in the art upon reading this disclosure and so forth.

It is also to be understood that the terminology used herein is for the purpose of describing particular embodiments only and is not intended to be limiting. Further, unless defined otherwise, all technical and scientific terms used herein have the same meaning as commonly understood by one of ordinary skill in the art to which this disclosure pertains. In describing and claiming the methods, systems, and computer readable media, the following terminology, and grammatical variants thereof, will be used in accordance with the definitions set forth below.

About: As used herein, “about” or “approximately” or “substantially” as applied to one or more values or elements of interest, refers to a value or element that is similar to a stated reference value or element. In certain embodiments, the term “about” or “approximately” or “substantially” refers to a range of values or elements that falls within 25%, 20%, 19%, 18%, 17%, 16%, 15%, 14%, 13%, 12%, 11%, 10%, 9%, 8%, 7%, 6%, 5%, 4%, 3%, 2%, 1%, or less in either direction (greater than or less than) of the stated reference value or element unless otherwise stated or otherwise evident from the context (except where such number would exceed 100% of a possible value or element).

Data set: As used herein, “data set” refers to a group or collection of information, values, or data points related to or associated with one or more objects, records, and/or variables. In some embodiments, a given data set is organized as, or included as part of, a matrix or tabular data structure. In some embodiments, a data set is encoded as a feature vector corresponding to a given image, object, record, and/or variable, such as a given test or reference subject. For example, a medical data set for a given subject can include one or more observed values of one or more variables associated with that subject.

Electronic neural network: As used herein, “electronic neural network” or “neural network” refers to a machine learning algorithm or model that includes layers of at least partially interconnected artificial neurons (e.g., perceptrons or nodes) organized as input and output layers with one or more intervening hidden layers that together form a network that is or can be trained to classify data, such as test subject medical data sets (e.g., image data sets or the like).

Machine Learning Algorithm: As used herein, “machine learning algorithm” generally refers to an algorithm, executed by computer, that automates analytical model building, e.g., for clustering, classification or pattern recognition. Machine learning algorithms may be supervised or unsupervised. Learning algorithms include, for example, artificial or electronic neural networks (e.g., back propagation networks), discriminant analyses (e.g., Bayesian classifier or Fisher's analysis), multiple-instance learning (MIL), support vector machines, decision trees (e.g., recursive partitioning processes such as CART-classification and regression trees, or random forests), linear classifiers (e.g., multiple linear regression (MLR), partial least squares (PLS) regression, and principal components regression), hierarchical clustering, and cluster analysis. A dataset on which a machine learning algorithm learns can be referred to as “training data.” A model produced using a machine learning algorithm is generally referred to herein as a “machine learning model.”

Resting state functional magnetic resonance imaging: As used herein, “resting state functional magnetic resonance imaging” or “rsfMRI” refers to a method of functional magnetic resonance imaging (fMRI) that is used in brain mapping to evaluate regional interactions that occur in a resting or task-negative state, when an explicit task is not being performed.

Subject: As used herein, “subject” or “test subject” refers to an animal, such as a mammalian species (e.g., human) or avian (e.g., bird) species. More specifically, a subject can be a vertebrate, e.g., a mammal such as a mouse, a primate, a simian or a human. Animals include farm animals (e.g., production cattle, dairy cattle, poultry, horses, pigs, and the like), sport animals, and companion animals (e.g., pets or support animals). A subject can be a healthy individual, an individual that has or is suspected of having a disease or pathology or a predisposition to the disease or pathology, or an individual that is in need of therapy or suspected of needing therapy. The terms “individual” or “patient” are intended to be interchangeable with “subject.” A “reference subject” refers to a subject known to have or lack specific properties (e.g., a known pathology, such as glioblastoma and/or the like).

System: As used herein, “system” in the context of analytical instrumentation refers a group of objects and/or devices that form a network for performing a desired objective.

Task based functional magnetic resonance imaging: As used herein, “task based functional magnetic resonance imaging” or “tfMRI” refers to a brain imaging modality that reveals functional activity of the brain to study the effects of a brain disease or disorder.

Treat. As used herein the terms “treat”, “treated”, or “treating” refer to both therapeutic treatment and prophylactic or preventative measures, wherein the object is to protect against (partially or wholly) or slow down (e.g., lessen or postpone the onset of) an undesired physiological condition, disorder or disease, or to obtain beneficial or desired clinical results such as partial or total restoration or inhibition in decline of a parameter, value, function or result that had or would become abnormal. For the purposes of this application, beneficial or desired clinical results include, but are not limited to, alleviation of symptoms; diminishment of the extent or vigor or rate of development of the condition, disorder or disease; stabilization (i.e., not worsening) of the state of the condition, disorder or disease; delay in onset or slowing of the progression of the condition, disorder or disease; amelioration of the condition, disorder or disease state; and remission (whether partial or total), whether or not it translates to immediate lessening of actual clinical symptoms, or enhancement or improvement of the condition, disorder or disease. Treatment seeks to elicit a clinically significant response without excessive levels of side effects.

### DETAILED DESCRIPTION

### Introduction

In some aspects, the present disclosure utilizes model-free seed selection in the vicinity of (i.e., proximal to) a brain tumor based on deep learning-based tumor tissue segmentation in combination with iterative subject-specific seed-optimization integrated into a real-time resting-state fMRI (rsfMRI) analysis pipeline to characterize peri-tumoral connectivity in patients with glioblastomas as part of treatment pathways for those patients. The methods of the present disclosure improve the specificity of peri-tumoral seed selection and automate seed placement in the vicinity of the tumor in the zone that is at risk during surgical resection without relying on, for example, neurofunctional brain atlases. These and other attributes will be apparent upon a complete review of the present disclosure, including the accompanying figures.

### Exemplary Methods

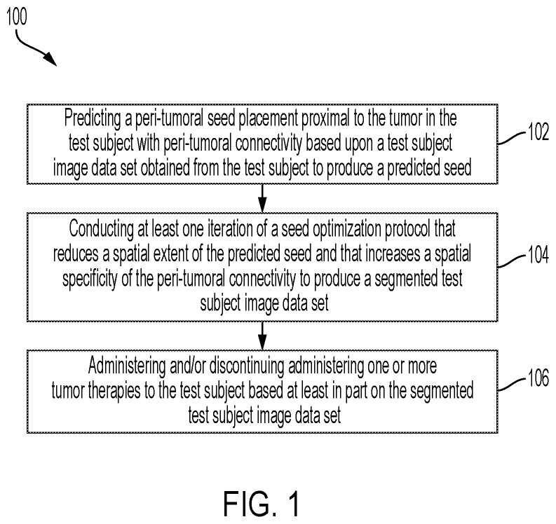

To illustrate, FIG. 1 is a flow chart that schematically shows exemplary method steps of treating a tumor in a test subject according to some aspects disclosed herein. As shown, method 100 includes predicting (e.g., by a computer) a peri-tumoral seed placement proximal to the tumor in the test subject with peri-tumoral connectivity based upon a test subject image data set obtained from the test subject to produce a predicted seed (step 102). In some embodiments, the test subject image data set comprises a magnetic resonance imaging (MRI) data set, such as a rsfMRI data set. In some embodiments, a cranium of the test subject comprises the tumor and the tumor comprises a glioblastoma. Method 100 also includes conducting (e.g., by the computer) at least one iteration of a seed optimization protocol that reduces a spatial extent of the predicted seed and that increases a spatial specificity of the peri-tumoral connectivity to produce a segmented test subject image data set (step 104). In some embodiments, the seed optimization protocol comprises a machine learning technique that involves the use of an electronic neural network. In some embodiments, the seed optimization protocol comprises a model-free seed selection process (e.g., does not utilize a neurofunctional brain atlas). In addition, method 100 also includes administering and/or discontinuing administering one or more tumor therapies to the test subject based at least in part on the segmented test subject image data set (step 106).

### Exemplary Systems and Computer Readable Media

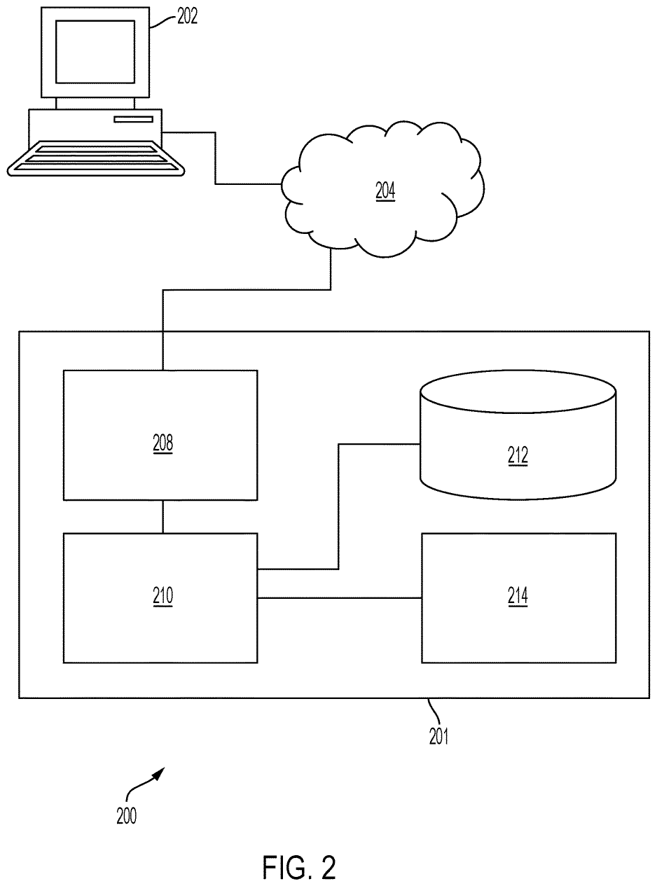

The present disclosure also provides various systems and computer program products or machine readable media. For example, FIG. 2 is a schematic diagram of a hardware computer system 200 suitable for implementing various embodiments. For example, FIG. 2 illustrates various hardware, software, and other resources that can be used in implementations of any of methods disclosed herein, including method 100 and/or one or more instances of an electronic neural network. System 200 includes training corpus source 202 and computer 201. Training corpus source 202 and computer 201 may be communicatively coupled by way of one or more networks 204, e.g., the internet.

Training corpus source 202 may include an electronic clinical records system, such as an LIS, a database, a compendium of clinical data, or any other source of test and/or reference subject image data sets (e.g, MRI data sets) suitable for use as a training corpus as disclosed herein. According to some embodiments, each component is implemented as a vector, such as a feature vector, that represents a respective tile. Thus, the term “component” refers to both a tile and a feature vector representing a tile.

Computer 201 may be implemented as any of a desktop computer, a laptop computer, can be incorporated in one or more servers, clusters, or other computers or hardware resources, or can be implemented using cloud-based resources. Computer 201 includes volatile memory 214 and persistent memory 212, the latter of which can store computer-readable instructions, that, when executed by electronic processor 210, configure computer 201 to perform any of the methods disclosed herein, including method 100, and/or form or store any electronic neural network, and/or perform any classification technique as described herein. Computer 201 further includes network interface 208, which communicatively couples computer 201 to training corpus source 202 via network 204. Other configurations of system 200, associated network connections, and other hardware, software, and service resources are possible.

Certain embodiments can be performed using a computer program or set of programs. The computer programs can exist in a variety of forms both active and inactive. For example, the computer programs can exist as software program(s) comprised of program instructions in source code, object code, executable code or other formats; firmware program(s), or hardware description language (HDL) files. Any of the above can be embodied on a transitory or non-transitory computer readable medium, which include storage devices and signals, in compressed or uncompressed form. Exemplary computer readable storage devices include conventional computer system RAM (random access memory), ROM (read-only memory), EPROM (erasable, programmable ROM), EEPROM (electrically erasable, programmable ROM), and magnetic or optical disks or tapes.

### Example: Tumor Tissue Segmentation for Seed-Based Mapping of Peritumoral Resting-State Connectivity

### Methods

RsfMRI (eyes open, 5-10 min) and tfMRI (3 min per task) data were acquired in 6 patients (4M,2F, 46-67 y) with glioblastomas using a 3T scanner equipped with 32-channel head coil. Informed written consent was obtained. Tasks were tailored to tumor location: motor (finger-tapping/fist-clenching), visual (eyes open/close), auditory (listening to syllables) and language (word/verb generation). Real-time fMRI was performed using single and multi-echo MB-EPI (TR/TE: 400/35 ms or TR/TE1/TE2: 400/15/41 ms, multiband-acceleration: 8, flip-angle: 30°, voxel-size: (3 mm)³, 40 slices). Real-time tfMRI and rsfMRI SBC analyses of major RSNs were performed using the TurboFIRE tool. RSNs were mapped using unilateral Brodmann area (BA) based seed regions (sensorimotor network (SMN): BA01-03, language networks (LAN): BA44,45 (Broca's) and BA22,39,40 (Wernicke's).

Peritumoral connectivity was measured offline by integrating deep learning-based tumor tissue segmentation into TurboFIRE. A 3-dimensional U-net convolutional neural network (CNN) based on the self-adaptive and benchmark-leading nnUNet method was used for glioma tumor subregion tissue prediction of the BraTS 2021 database. The network is based on a 128×128×128 1-mm³ isotropic voxel patch with sliding window inference. Data augmentation during both training and testing was used with a 6-layer UNet containing 31 million trainable weights. Training and cross-validation were performed using k-fold cross validation (k=5). Training and predictions employed an NVIDIA 3090 GPU. A client-server Representational State Transfer (REST) HTTP interface for the trained CNN was integrated into the TurboFIRE real-time fMRI analysis tool to perform segmentation of enhancing tumor, non-enhancing tumor/necrotic core, and edema in FLAIR images. Skull stripping was not required, since peripheral regions have low intensity in fMRI. The tumor tissue mask (viable tumor and necrotic core) was dilated by a fixed distance, an eroded tumor tissue mask was subtracted and the union of peritumoral edema was added to generate a peritumor eloquent tissue area for initial seeding and sliding-window correlation analysis. Subject specific seed-optimization was performed using (a) thresholding of the cumulative meta-mean maps and (b) a cluster analysis to locate contiguous above-threshold voxels within the original seed regions to form the seed regions for the subsequent iteration.

Peritumoral connectivity was measured offline by integrating deep learning-based tumor tissue segmentation into TurboFIRE. A 3-dimensional U-net convolutional neural network (CNN) based on the nnUNet method trained with the BraTS 2021 dataset was used for glioma tumor subregion tissue prediction. The BraTS dataset labels tumor subregions with 3 non-overlapping classes: NET (non-enhancing tumor), ED (peritumoral edema), and ET (enhancing tumor). The 6 layer, 31 million trainable weight CNN uses a 128×128×128 1-mm³ isotropic voxel patch for training and sliding window inference. Data augmentation was employed for both training and prediction. The loss function uses an average of cross-entropy and dice coefficient based over the overlapping labels used for scoring the BraTS competition: whole tumor (WT=NET+ED+ET), tumor core (TC=NET+ET), and edema (ED). Training and cross-validation were performed using k-fold cross validation (k=5) and employed an NVIDIA 3090 GPU. While the BraTS dataset contains four MRI contrasts (FLAIR, T1, T1CE, T2), our subjects had a single T2 contrast. To assess the effectiveness of predicting with less than all 4 MRI BraTS contrasts, models were trained with all possible subsets of the MRI contrasts with a total of 75 models evaluated (15 combinations of MRI contrasts with 5 kfolds each).

### Results

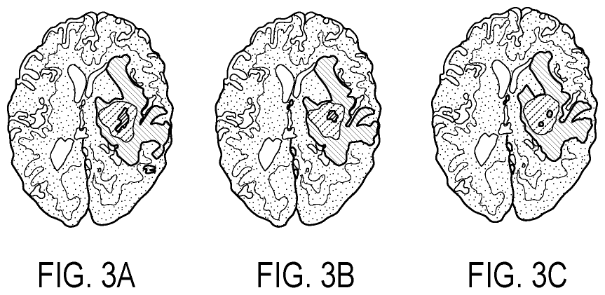

Mean cross-validation Dice similarity scores (Table 1) with 1251 subjects using k-fold training and validation with k=5 employing four MRI contrasts (T₁, T₁CE, T₂, FLAIR) were 0.860±0.170 (FIG. 3B) and using only T₂ contrast were 0.678±0.213 (FIG. 3C). The four contrast DICE scores were similar to the results in another study. Subregion DICE scores of edema for T₂ only (0.788±0.148) were the closest to those for four MRI contrasts (0.885±0.112). The time for predicting tumor segmentation with four MRI contrasts and using only the T₂ MRI was 23 s.

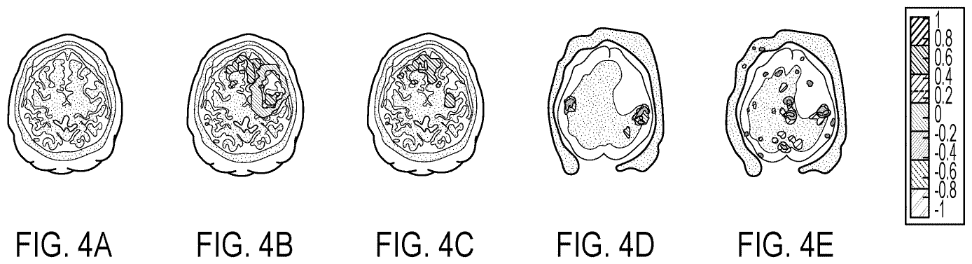

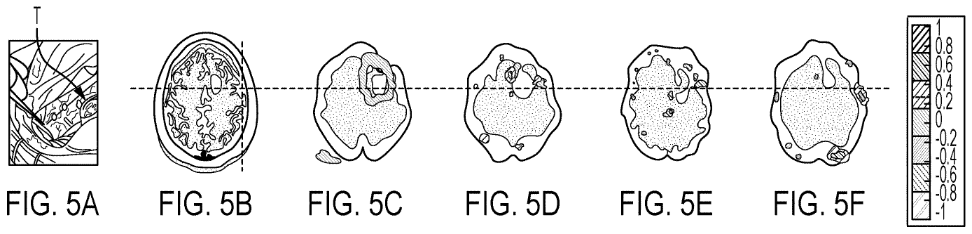

FIG. 4 shows an example of tissue prediction based seeding in comparison with sensorimotor rsfMRI and motor tfMRI in a patient with a glioblastoma using the dilated tumor tissue mask revealed unanticipated connectivity between an area posterior to the tumor and frontal cortex (FIG. 4A) that became spatially more constrained when using iterative seed optimization (FIG. 4B). This connectivity was spatially segregated from resting-state motor connectivity (FIG. 4C) and task-based motor activation (FIG. 4D). Intra-operatively, resection was subtotal at the posterior and anterior margins due to concerns for functional deficits. Peri-tumoral connectivity in another patient with a glioblastoma (FIG. 5) was detected lateral and medial to the tumor (FIG. 3D), which was in close proximity to Exner's area that was detected using language resting-state connectivity (FIG. 5E) and task-activation (FIG. 5F) as shown in our earlier work.

TABLE 1
Dice crossvalidation based on 1251 subjects in the BraTS database using four
MRI contrasts (T₁, T₁CE, T₂, FLAIR) and single T₂ contrast averaged across k-fold
training. NET = nonenhancing tumor + necrotic tissue, ED = edema, ET = enhancing
tumor.

| | | | |
|---|---|---|---|
| L1 (NET) | L2 (ED) | L4 (ET) | Mean |

Four MRI contrasts

| | | | | |
|---|---|---|---|---|
| Mean (k = 0.4) | 0.78912 | 0.88464 | 0.90478 | 0.8595 |
| SD (k = 0.4) | 0.26332 | 0.1124 | 0.13312 | 0.1696 |

T2 Only

| | | | | |
|---|---|---|---|---|
| Mean (k = 0.4) | 0.524 | 0.788 | 0.722 | 0.678 |
| SD (k = 0.4) | 0.3028 | 0.14772 | 0.18704 | 0.21252 |

Mean dice similarity scores for non-overlapping (Table 2) and overlapping labels (Table 3) with 1251 subjects were highest with using all 4 MRI contrasts (83.80 and 91.15) and lowest when using T1 alone (57.99 and 74.8). With T2 alone as used with our subjects, mean dice scores were 63.45 and 77.89. The best performing single MRI contrast was T1CE with mean dice scores of 78.72 and 87.85. The best performing combination of two MRI contrasts was FLAIR+T1CE with mean dice scores of 83.16 and 90.66.

TABLE 2
Non-overlapping labels

| Contrasts | Mean | NET | ED | ET |
|---|---|---|---|---|
| FLAIR | 62.55 ± 23.20 | 44.73 ± 30.51 | 79.23 ± 15.86 | 63.69 ± 23.23 |
| T1 | 57.99 ± 24.20 | 45.24 ± 29.95 | 67.28 ± 19.92 | 61.44 ± 22.73 |
| T1CE | 78.72 ± 21.72 | 75.63 ± 28.91 | 73.24 ± 19.03 | 87.29 ± 17.20 |
| T2 | 63.45 ± 23.07 | 50.14 ± 29.78 | 75.44 ± 16.99 | 64.76 ± 22.43 |
| FLAIR + T1 | 67.18 ± 22.74 | 53.84 ± 30.11 | 80.16 ± 15.79 | 67.52 ± 22.32 |
| FLAIR + T1CE | 83.16 ± 19.77 | 76.24 ± 28.65 | 85.44 ± 13.85 | 87.80 ± 16.81 |
| FLAIR + T2 | 68.19 ± 22.72 | 54.84 ± 30.36 | 80.78 ± 15.63 | 68.95 ± 22.16 |
| T1 + T1CE | 79.46 ± 21.33 | 75.91 ± 28.73 | 74.69 ± 18.63 | 87.77 ± 16.65 |
| T1 + T2 | 65.19 ± 22.94 | 52.84 ± 29.84 | 76.46 ± 16.77 | 66.26 ± 22.20 |
| T1CE + T2 | 81.71 ± 20.28 | 75.93 ± 28.72 | 81.49 ± 15.32 | 87.71 ± 16.81 |
| FLAIR + T1 + T1CE | 83.30 ± 19.53 | 76.10 ± 28.14 | 85.63 ± 13.94 | 88.18 ± 16.52 |
| FLAIR + T1 + T2 | 69.30 ± 22.69 | 57.71 ± 29.87 | 80.89 ± 15.87 | 69.29 ± 22.33 |
| FLAIR + T1CE + T2 | 83.50 ± 19.56 | 76.33 ± 28.16 | 86.23 ± 13.86 | 87.94 ± 16.67 |
| T1 + T1CE + T2 | 82.06 ± 20.07 | 76.19 ± 28.45 | 81.82 ± 15.42 | 88.16 ± 16.33 |
| FLAIR + T1 + T1CE + T2 | 83.80 ± 19.43 | 76.82 ± 28.14 | 86.17 ± 13.99 | 88.42 ± 16.15 |

TABLE 3
Overlapping labels

| | Mean | WT | TC | ET |
|---|---|---|---|---|
| FLAIR | 78.43 ± 17.27 | 92.12 ± 8.374 | 79.48 ± 20.22 | 63.70 ± 23.20 |
| T1 | 74.81 ± 18.72 | 85.24 ± 12.75 | 77.82 ± 20.65 | 61.36 ± 22.76 |
| T1CE | 87.85 ± 15.09 | 85.81 ± 12.23 | 90.81 ± 15.10 | 86.92 ± 17.93 |
| T2 | 77.89 ± 17.27 | 89.95 ± 9.134 | 79.13 ± 20.15 | 64.60 ± 22.53 |
| FLAIR + T1 | 80.44 ± 16.58 | 92.47 ± 8.176 | 81.48 ± 19.12 | 67.37 ± 22.43 |
| FLAIR + T1CE | 90.66 ± 12.97 | 93.04 ± 7.368 | 91.53 ± 13.98 | 87.43 ± 17.56 |
| FLAIR + T2 | 81.33 ± 15.96 | 92.85 ± 7.520 | 82.18 ± 18.27 | 68.98 ± 22.09 |
| T1 + T1CE | 88.46 ± 14.59 | 86.66 ± 11.89 | 91.29 ± 14.57 | 87.42 ± 17.32 |
| T1 + T2 | 78.98 ± 16.93 | 90.38 ± 9.152 | 80.44 ± 19.32 | 66.10 ± 22.31 |
| T1CE + T2 | 89.86 ± 13.41 | 90.89 ± 8.068 | 91.16 ± 15.03 | 87.51 ± 17.14 |
| FLAIR + T1 + T1CE | 90.96 ± 12.79 | 93.21 ± 7.428 | 91.71 ± 13.98 | 87.96 ± 16.95 |
| FLAIR + T1 + T2 | 81.43 ± 16.35 | 92.82 ± 8.237 | 82.36 ± 18.37 | 69.13 ± 22.46 |
| FLAIR + T1CE + T2 | 90.97 ± 12.79 | 93.56 ± 7.240 | 91.88 ± 13.46 | 87.47 ± 17.65 |
| T1 + T1CE + T2 | 90.18 ± 13.06 | 91.02 ± 8.376 | 91.71 ± 13.76 | 87.80 ± 17.05 |
| FLAIR + T1 + T1CE + T2 | 91.15 ± 12.75 | 93.53 ± 7.431 | 91.96 ± 13.68 | 87.96 ± 17.14 |

### Discussion

This model-free seeding approach enables systematic assessment of peri-tumoral resting-state connectivity and cortical eloquence in different tumor subregions, such as edematous and infiltrative regions. It addresses limitations of machine learning-based seed selection in the vicinity of slow-growing and infiltrative lesions with major changes in functional neuroanatomy. The feasibility of using single-contrast MRI for predicting edematous regions facilitates the integration of automated real-time resting-state fMRI into clinical settings, such as intra-operative fMRI, where time constraints may limit the number of MRI contrasts. Intra-operative ECS of peri-tumoral regions, more detailed task-based mapping and DTI-based tractography can be used to further characterize the neurofunctional correlates of peri-tumoral connectivity.

### CONCLUSIONS

Seed selection based on tissue prediction in combination with seed optimization provides an unbiased approach for probing peri-lesional resting-state fMRI connectivity in patients with brain tumors. The developed tissue prediction based seeding approach enhances presurgical assessment of surgical risk in the vicinity of eloquent cortex.

Some further aspects are defined in the following clauses:
Clause 1: A method of treating a tumor in a test subject, the method comprising: predicting, by a computer, a peri-tumoral seed placement proximal to the tumor in the test subject with peri-tumoral connectivity based upon a test subject image data set obtained from the test subject to produce a predicted seed; conducting, by the computer, at least one iteration of a seed optimization protocol that reduces a spatial extent of the predicted seed and that increases a spatial specificity of the peri-tumoral connectivity to produce a segmented test subject image data set; and, administering and/or discontinuing administering one or more tumor therapies to the test subject based at least in part on the segmented test subject image data set, thereby treating the tumor in the subject.
Clause 2: The method of Clause 1, wherein the predicting step comprises segmenting tissue to identify tumor subregions and tumor periphery for the seed placement.
Clause 3: The method of Clause 1 or Clause 2, wherein the test subject image data set comprises a magnetic resonance imaging (MRI) data set.
Clause 4: The method of any one of the preceding Clauses 1-3, wherein the MRI data set comprises a rsfMRI data set.
Clause 5: The method of any one of the preceding Clauses 1-4, wherein the seed optimization protocol comprises a model-free seed selection process.
Clause 6: The method of any one of the preceding Clauses 1-5, wherein the seed optimization protocol comprises a machine learning technique.
Clause 7: The method of any one of the preceding Clauses 1-6, wherein a cranium of the test subject comprises the tumor.
Clause 8: The method of any one of the preceding Clauses 1-7, comprising using the test subject image data set and/or the segmented test subject image data set as a basis for identifying eloquent cortex in the test subject as a guide to treatment of the test subject.
Clause 9: The method of any one of the preceding Clauses 1-8, wherein the tumor comprises a glioblastoma.
Clause 10: The method of any one of the preceding Clauses 1-9, wherein the method does not utilize a neurofunctional brain atlas.
Clause 11: A system for producing a tumor therapy recommendation for a test subject having a tumor using a test subject image data set obtained from the test subject, the system comprising: a processor; and a memory communicatively coupled to the processor, the memory storing instructions which, when executed on the processor, perform operations comprising: predicting a peri-tumoral seed placement proximal to the tumor in the test subject with peri-tumoral connectivity based upon the test subject image data set obtained from the test subject to produce a predicted seed; conducting at least one iteration of a seed optimization protocol that reduces a spatial extent of the predicted seed and that increases a spatial specificity of the peri-tumoral connectivity to produce a segmented test subject image data set; and, generating a tumor therapy recommendation report that comprises one or more recommendations to administer and/or discontinue administering one or more tumor therapies to the test subject based at least in part on the segmented test subject image data set.
Clause 12: The system of Clause 11, wherein the test subject image data set comprises a magnetic resonance imaging (MRI) data set.
Clause 13: The system of Clause 11 or Clause 12, wherein the seed optimization protocol comprises a model-free seed selection process.
Clause 14: The system of any one of the preceding Clauses 8-13, wherein the seed optimization protocol and/or tissue prediction comprises a machine learning technique.
Clause 15: The system of any one of the preceding Clauses 8-14, wherein the seed optimization protocol utilizes an electronic neural network.
Clause 16: The system of any one of the preceding Clauses 8-15, wherein a cranium of the test subject comprises the tumor.
Clause 17: The system of any one of the preceding Clauses 8-16, wherein the tumor comprises a glioblastoma.
Clause 18: The system of any one of the preceding Clauses 8-17, wherein the system does not utilize a neurofunctional brain atlas.
Clause 19: A computer readable media comprising non-transitory computer executable instructions which, when executed by at least one electronic processor, perform at least: predicting a peri-tumoral seed placement proximal to tumor in a test subject with peri-tumoral connectivity based upon a test subject image data set obtained from the test subject to produce a predicted seed; conducting at least one iteration of a seed optimization protocol that reduces a spatial extent of the predicted seed and that increases a spatial specificity of the peri-tumoral connectivity to produce a segmented test subject image data set; and, generating a tumor therapy recommendation report that comprises one or more recommendations to administer and/or discontinue administering one or more tumor therapies to the test subject based at least in part on the segmented test subject image data set.
Clause 20: The computer readable media of Clause 19, wherein the test subject image data set comprises a magnetic resonance imaging (MRI) data set.
Clause 21: The computer readable media of Clause 19 or Clause 20, wherein the seed optimization protocol comprises a model-free seed selection process.
Clause 22: The computer readable media of any one of the preceding Clauses 19-21, wherein the seed optimization protocol comprises a machine learning technique.
Clause 23: The computer readable media of any one of the preceding Clauses 19-22, wherein a cranium of the test subject comprises the tumor.
Clause 24: The computer readable media of any one of the preceding Clauses 19-23, wherein the tumor comprises a glioblastoma.
Clause 25: The computer readable media of any one of the preceding Clauses 19-24, wherein the computer readable media does not utilize a neurofunctional brain atlas.

Although this disclosure contains many specific embodiment details, these should not be construed as limitations on the scope of the subject matter or on the scope of what may be claimed, but rather as descriptions of features that may be specific to particular embodiments. Certain features that are described in this disclosure in the context of separate embodiments can also be implemented, in combination, in a single embodiment. Conversely, various features that are described in the context of a single embodiment can also be implemented in multiple embodiments, separately, or in any suitable sub-combination. Moreover, although previously described features may be described as acting in certain combinations and even initially claimed as such, one or more features from a claimed combination can, in some cases, be excised from the combination, and the claimed combination may be directed to a sub-combination or variation of a sub-combination.

Particular embodiments of the subject matter have been described. Other embodiments, alterations, and permutations of the described embodiments are within the scope of the following claims as will be apparent to those skilled in the art. While operations are depicted in the drawings or claims in a particular order, this should not be understood as requiring that such operations be performed in the particular order shown or in sequential order, or that all illustrated operations be performed (some operations may be considered optional), to achieve desirable results.

Accordingly, the previously described example embodiments do not define or constrain this disclosure. Other changes, substitutions, and alterations are also possible without departing from the spirit and scope of this disclosure.

1. A method of treating a tumor in a test subject, the method comprising:
predicting, by a computer, a peri-tumoral seed placement proximal to the tumor in the test subject with peri-tumoral connectivity based upon a test subject image data set obtained from the test subject to produce a predicted seed;
conducting, by the computer, at least one iteration of a seed optimization protocol that reduces a spatial extent of the predicted seed and that increases a spatial specificity of the peri-tumoral connectivity to produce a segmented test subject image data set; and,
administering and/or discontinuing administering one or more tumor therapies to the test subject based at least in part on the segmented test subject image data set, thereby treating the tumor in the subject.

2. The method of claim 1, wherein the predicting step comprises segmenting tissue to identify tumor subregions and tumor periphery for the seed placement.

3. The method of claim 1, wherein the test subject image data set comprises a magnetic resonance imaging (MRI) data set.

4. The method of claim 3, wherein the MRI data set comprises a rsfMRI data set.

5. The method of claim 1, wherein the seed optimization protocol comprises a model-free seed selection process.

6. The method of claim 1, wherein the seed optimization protocol comprises a machine learning technique.

7. The method of claim 1, wherein a cranium of the test subject comprises the tumor.

8. The method of claim 7, comprising using the test subject image data set and/or the segmented test subject image data set as a basis for identifying eloquent cortex in the test subject as a guide to treatment of the test subject.

9. The method of claim 1, wherein the tumor comprises a glioblastoma.

10. The method of claim 1, wherein the method does not utilize a neurofunctional brain atlas.

11. A system for producing a tumor therapy recommendation for a test subject having a tumor using a test subject image data set obtained from the test subject, the system comprising:
a processor; and
a memory communicatively coupled to the processor, the memory storing instructions which, when executed on the processor, perform operations comprising:
predicting a peri-tumoral seed placement proximal to the tumor in the test subject with peri-tumoral connectivity based upon the test subject image data set obtained from the test subject to produce a predicted seed;
conducting at least one iteration of a seed optimization protocol that reduces a spatial extent of the predicted seed and that increases a spatial specificity of the peri-tumoral connectivity to produce a segmented test subject image data set; and,
generating a tumor therapy recommendation report that comprises one or more recommendations to administer and/or discontinue administering one or more tumor therapies to the test subject based at least in part on the segmented test subject image data set.

12. (canceled)

13. The system of claim 11, wherein the seed optimization protocol comprises a model-free seed selection process.

14. The system of claim 11, wherein the seed optimization protocol and/or tissue prediction comprises a machine learning technique.

15. (canceled)

16. The system of claim 11, wherein a cranium of the test subject comprises the tumor.

17. The system of claim 11, wherein the tumor comprises a glioblastoma.

18. The system of claim 11, wherein the system does not utilize a neurofunctional brain atlas.

19. A computer readable media comprising non-transitory computer executable instructions which, when executed by at least one electronic processor, perform at least:
predicting a peri-tumoral seed placement proximal to tumor in a test subject with peri-tumoral connectivity based upon a test subject image data set obtained from the test subject to produce a predicted seed;
conducting at least one iteration of a seed optimization protocol that reduces a spatial extent of the predicted seed and that increases a spatial specificity of the peri-tumoral connectivity to produce a segmented test subject image data set; and,
generating a tumor therapy recommendation report that comprises one or more recommendations to administer and/or discontinue administering one or more tumor therapies to the test subject based at least in part on the segmented test subject image data set.

20. (canceled)

21. The computer readable media of claim 19, wherein the seed optimization protocol comprises a model-free seed selection process.

22. (canceled)

23. (canceled)

24. The computer readable media of claim 19, wherein the tumor comprises a glioblastoma.

25. The computer readable media of claim 19, wherein the computer readable media does not utilize a neurofunctional brain atlas.
